(19) United States
(12) Patent Application Publication
(10) Pub. No.: US 2018/0192855 A1
(43) Pub. Date: Jul. 12, 2018

(54) INSERTION ASSIST SYSTEM AND INSERTION INSTRUMENT

(71) Applicant: OLYMPUS CORPORATION, Tokyo (JP)

(72) Inventor: Yuji SAKAI, Kodaira-shi (JP)

(73) Assignee: OLYMPUS CORPORATION, Tokyo (JP)

(21) Appl. No.: 15/404,567

(22) Filed: Jan. 12, 2017

(51) Int. Cl. A61B 1/00

(52) U.S. Cl. CPC A61B 1/00154; A61B 1/00167; A61B 1/00158

(57) ABSTRACT

An insertion assist system includes an insertion instrument and an assist instrument. The insertion instrument includes an elongated member, a first engagement portion provided in the elongated member, and a rigid portion provided on a distal side of the elongated member and having an appropriate length. The assist instrument includes a guide path that includes a bent section, at least apart of the bent section being bent, and that is configured to guide the rigid portion of the insertion instrument to a desired position on a distal side from the distal section, and a second engagement portion that is provided in the guide path and engaged with the first engagement portion of the insertion instrument, and that maintains the elongated member and the guide path in a contact or close state.

### BACKGROUND OF THE INVENTION

### 1. Field of the Invention

The present invention relates to an insertion assist system and an insertion instrument.

### 2. Description of the Related Art

Jpn. Pat. Appln. KOKAI Publication No. 2003-159257 discloses, for example, a treatment instrument including at a distal end a rigid portion that is guided outside a straight cylindrical body along a longitudinal axis.

For example, U.S. Pat. No. 6,878,106 B1 discloses a treatment instrument guided along an outside of a bent fiber scope.

### BRIEF SUMMARY OF THE INVENTION

According to one aspect of the present invention, an insertion assist system includes: an insertion instrument including an elongated member having flexibility and extending along a longitudinal axis, a first engagement portion provided in the elongated member, and a rigid portion provided on a distal side of the elongated member and having an appropriate length; and an assist instrument including a guide path that includes a distal section, and a bent section, at least a part of the bent section being bent at a position on a proximal side from the distal section, and that is configured to guide the rigid portion of the insertion instrument to a desired position on a distal side from the distal section, and a second engagement portion that is provided in the guide path and engaged with the first engagement portion of the insertion instrument, and that maintains the elongated member and the guide path in a contact or close state.

According to another aspect of the present invention, an insertion assist system includes: an insertion instrument including: an insertion main body including a flexible tube having flexibility and extending along a longitudinal axis, and a rigid portion provided on a distal side of the flexible tube and having an appropriate length, a cylindrical body through which the insertion main body is inserted, and which is provided on an outer circumferential surface of the insertion main body and forms an elongated member having an appropriate elastic toughness in association with the flexible tube, and a first engagement portion provided at a position on a proximal side from the rigid portion in the cylindrical body in a state in which a distal section of the insertion main body is arranged at a distal end of the cylindrical body; and an assist instrument including a guide path that includes a distal section and a bent section, at least a part of the bent section being bent at a position on a proximal side from the distal section, and that is configured to guide the rigid portion to a desired position on a distal side from the distal section, the assist instrument including a second engagement portion that is provided in the guide path and engaged with the first engagement portion, and maintains the cylindrical body and the guide path in a contact or close state.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute apart of the specification, illustrate embodiments of the invention, and together with the general description given above and the detailed description of the embodiments given below, serve to explain the principles of the invention.

FIG. 1 is a schematic view showing a treatment system including an insertion assist system according to a first embodiment and a second embodiment.

FIG. 2 is a schematic view showing an endoscope as an example of an insertion instrument to be inserted through an assist instrument of the insertion assist system according to the first embodiment and the second embodiment, in which apart near a distal section of an insertion section is enlarged relative to a proximal section of the insertion section.

FIG. 3A is a schematic view showing a state in which a first engagement portion of the insertion instrument is engaged with a second engagement portion of the assist instrument of the insertion assist system according to the first embodiment, while the insertion instrument is moved relative to the assist instrument, so that a distal end of the insertion instrument is located at a proximal section of a bent section of a pipe-shaped guide path of the assist instrument.

FIG. 3B is a schematic view showing a cross section taken along line 3B-3B in FIG. 3A.

FIG. 3C is a schematic view showing a cross section taken along line 3B-3B in FIG. 3A of an example other than that shown in FIG. 3B.

FIG. 4 is a schematic view showing a state in which the first engagement portion of the insertion instrument is engaged with the second engagement portion of the assist instrument of the insertion assist system according to the first embodiment, while the insertion instrument is moved relative to the assist instrument, so that the distal end of the insertion instrument is located at a distal section of the bent section of the guide path of the assist instrument.

FIG. 5 is a schematic view showing a state in which the first engagement portion of the insertion instrument is engaged with the second engagement portion of the assist instrument of the insertion assist system according to the first embodiment, while the insertion instrument is moved relative to the assist instrument, so that the distal end of the insertion instrument is located at the distal section of the guide path of the assist instrument.

FIG. 6A is a schematic view showing a state in which the first engagement portion of the insertion instrument is engaged with the second engagement portion of the assist instrument of the insertion assist system according to the first embodiment, while the insertion instrument is moved relative to the assist instrument, so that the distal end of the insertion instrument is projected to a predetermined position from the distal section of the guide path of the assist instrument directed upward.

FIG. 6B is a schematic view showing a state in which a slit is formed appropriately in the first engagement portion of the insertion instrument shown in FIG. 6A.

FIG. 6C is a schematic view showing a state in which a slit is formed appropriately in the second engagement portion of the insertion instrument shown in FIG. 6A.

FIG. 7 is a schematic view showing a state in which the first engagement portion of the insertion instrument is engaged with the second engagement portion of the assist instrument of the insertion assist system according to the first embodiment, while the insertion instrument is moved relative to the assist instrument, so that the distal end of the insertion instrument is projected to a predetermined position from the distal section of the guide path of the assist instrument directed downward.

FIG. 8A is a schematic view showing a state in which a first engagement portion of the insertion instrument is engaged with a second engagement portion of the assist instrument of the insertion assist system according to the second embodiment, while the insertion instrument is moved relative to the assist instrument, so that a distal end of the insertion instrument is located at a proximal section of a bent section of a half-pipe shaped or spatula shaped guide path of the assist instrument.

FIG. 8B is a schematic view showing a state in which the second engagement portion of the insertion instrument shown in FIG. 6A is formed in the guide path, not the base portion.

FIG. 9A is a schematic view showing a state in which the first engagement portion of the insertion instrument is engaged with the second engagement portion of the assist instrument of the insertion assist system according to the second embodiment, while the insertion instrument is moved relative to the assist instrument, so that the distal end of the insertion instrument is projected to a predetermined position from the distal section of the guide path of the assist instrument directed upward.

FIG. 9B is a schematic view showing a cross section taken along line 9B-9B in FIG. 9A.

FIG. 10 is a schematic view showing a state in which the first engagement portion of the insertion instrument is engaged with the second engagement portion of the assist instrument of the insertion assist system according to the second embodiment, while the insertion instrument is moved relative to the assist instrument, so that the distal end of the insertion instrument is projected in a predetermined position from the distal section of the guide path of the assist instrument directed downward.

FIG. 11A is a schematic view showing a cross section taken along line 9B-9B in FIG. 9A of an example other than that shown in FIG. 9B.

FIG. 11B is a schematic view showing a cross section taken along line 9B-9B in FIG. 9A of an example other than those shown in FIG. 9B and FIG. 11A.

### DETAILED DESCRIPTION OF THE INVENTION

Embodiments of the present invention will be described with reference to the drawings.

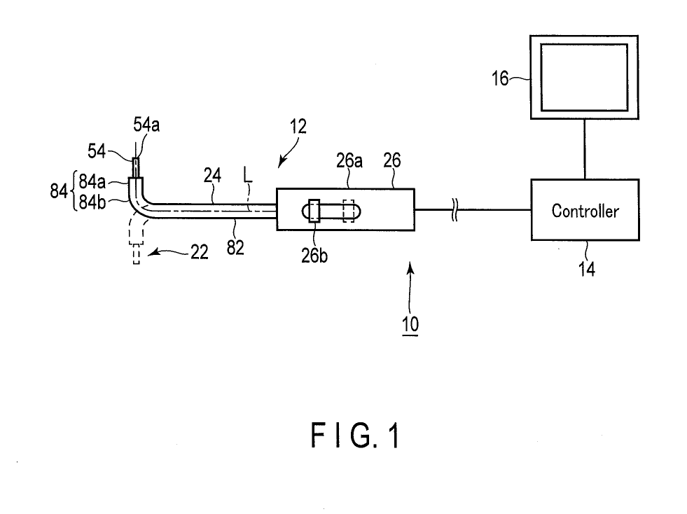

A first embodiment will be described with reference to FIG. 1 to FIG. 7.

A treatment system 10 shown in FIG. 1 includes an insertion assist system 12 and a controller 14.

The insertion assist system 12 is used to assist in guiding a rigid portion 54 of an insertion instrument 22 (to be described later) out of a distal section 84a of a guide path 84 to a predetermined location relative to the distal section 84a of the guide path 84. The insertion assist system 12 is used in a case of observing and treating, for example, a paranasal sinus, a bladder, a urinary organ, a bronchial tube, a pancreaticobiliary duct, etc., as needed. The insertion assist system 12 may be used for any other region as needed.

The controller 14 is selected depending on the intended use. For example, if the insertion instrument 22 is an endoscope, the controller 14 controls emission of appropriate light from a light source not shown, converts an image of a region facing the distal end of the insertion section of the endoscope to an electric signal, and causes the electric signal to be displayed in a display 16 shown in FIG. 1. For example, if the insertion instrument 22 is a monopolar or bipolar high-frequency treatment instrument, the controller 14 appropriately supplies energy to the treatment instrument. If the insertion instrument 22 is a treatment instrument, it may be an electrothermal treatment instrument using a heater, or an ultrasonic treatment instrument using ultrasonic vibrations. Thus, a variety of instruments may be used as the insertion instrument 22. The insertion instrument 22 is not necessarily formed as a single member, but may be formed as an insertion instrument assembly. As will be detailed later, an insertion instrument assembly using two members in combination, which are an endoscope 32 and a cylindrical body 34, is used as the insertion instrument 22.

The insertion assist system 12 includes the insertion instrument 22, an assist instrument 24 that assists in inserting the insertion instrument 22, and a handle 26. The handle 26 includes a handle main body 26a fixed to a proximal end of the assist instrument 24, and a movable portion 26b that is movable relative to the handle main body 26a and that moves the insertion instrument 22 relative to the assist instrument 24.

As described above, the insertion instrument 22 may be an appropriate treatment instrument, such as a high-frequency treatment instrument, or an endoscope to observe an inside of a body. In this embodiment, the insertion instrument 22 is described as an element including the endoscope (insertion main body) 32, and the cylindrical body (sheath) 34 covering a periphery of an insertion section 42 of the endoscope 32.

The insertion instrument 22 includes an elongated member having flexibility and extending along a longitudinal axis L, and a rigid portion 54 (to be described later) having an appropriate length and provided on a distal side of the elongated member. The elongated member here includes a flexible portion (flexible tube) 52 (to be described later) of the insertion section 42 of the endoscope 32, and a main body 34a (to be described later) of the cylindrical body 34. If no cylindrical body 34 is present, the elongated member is the flexible portion 52 (to be described later) of the insertion section 42 of the endoscope 32.

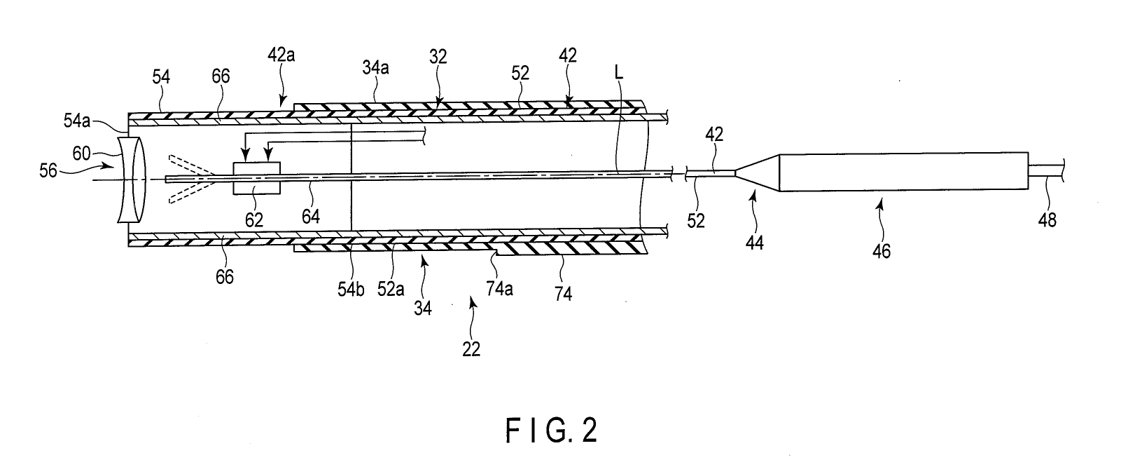

The insertion instrument 22, that is, the endoscope 32 and the cylindrical body 34 are attached to the assist instrument 24 when used. As shown in FIG. 2, the endoscope 32 includes the insertion section 42, a protection hood 44, a support section 46, and a cable 48. The distal end of the insertion section 42 is projectable from a distal section (distal opening) 84a (to be described later) of the assist instrument 24. The flexible portion (flexible tube) 52 and the rigid portion 54 of the endoscope 32 can move together with the cylindrical body 34 and can also move relative to the cylindrical body 34.

The endoscope 32 can capture an image of a region facing a distal end face 54a (to be described later) of the insertion section 42, and the image can be displayed in the display 16. The endoscope 32 may be of any type, for example, a fiber type or an image-pickup device type, such as a CCD or CMOS; however, in this embodiment, the endoscope is assumed to be of a scanning type.

Since the scanning-type endoscope 32 is conventionally known, detailed descriptions are omitted. A distal section 42a of the insertion section 42 has an internal structure as shown in FIG. 2. In the scanning-type endoscope 32, the diameter of the insertion section 42 can be smaller than that of a fiber type or an image-pickup device type. Therefore, the scanning-type endoscope 32 is suitable for insertion through a very narrow cavity, such as a paranasal sinus.

The insertion section 42 of the endoscope 32 includes the flexible portion (elongated member) 52 having flexibility and extending along the longitudinal axis L, the rigid portion 54 provided on a distal end side of the flexible portion 52, and an observation optical system 56. Outer diameters of the flexible portion 52 and the rigid portion 54 are both several millimeters. If the outer diameters of the flexible portion 52 and the rigid portion 54 are very small as in the case of the endoscope 32, it is difficult to form a bending mechanism in the flexible portion 52. Therefore, in this description, it is assumed that the flexible portion 52 is soft and is easily bent by an external force such as gravitation. It is also preferable that the flexible portion 52 has a function of actively bending in, for example, a distal section 52a.

The observation optical system 56 is arranged inside the insertion section 42. A distal section of the observation optical system 56 is fixed to the rigid portion 54. Thus, the rigid portion 54 has an appropriate length and rigidness, so that the rigid portion cannot easily be deformed by a reaction force when it is brought into contact with a living tissue. The rigid portion 54 is made of a rigid material, such as stainless steel, and its periphery is covered with a material having an electrical insulation property.

In the observation optical system 56, an actuator 62, an illuminating fiber 64, and a plurality of light-receiving fibers 66 are optically and/or electrically connected to the controller 14 shown in FIG. 1. The controller 14 shown in FIG. 1 controls the observation optical system 56 of the endoscope 32. The controller 14 controls operations of the actuator 62. The controller 14 includes a light source (not shown) of, for example, white light, and causes light for observation to be incident in the illuminating fiber 64 as appropriate. The controller 14 creates an image from the light received by the light-receiving fibers 66.

An illumination window 60, the actuator 62 provided on a proximal side of the illumination window 60, a distal section of the illuminating fiber 64, and distal sections of the light-receiving fibers 66 are arranged within the rigid portion 54. The illumination window 60 and distal ends of the light-receiving fibers 66 are fixed to the distal end face (distal end) 54a of the rigid portion 54. The distal ends of the light-receiving fibers 66 are fixed to a periphery of the illumination window 60 at appropriate intervals.

The flexible portion 52 is arranged on a proximal side of the rigid portion 54. The flexible portion 52 extends from the proximal end of the rigid portion 54 to the proximal side. Almost the entire length of the insertion section 42 is formed of the flexible portion 52 as a part having flexibility. The protection hood 44 is fixed to a proximal end of the flexible portion 52. The support section 46 is fixed to a proximal end of the protection hood 44. The cable 48 is fixed to a proximal end of the support section 46. A proximal end of the cable 48 is connected to the controller 14. The support section 46 is connected to the movable portion 26b of the handle 26. Therefore, when the movable portion 26b is moved relative to the handle main body 26a along the longitudinal axis L, the distal end of the insertion section 42 of the endoscope 32 can be projected from or retracted in the distal section 84a of the assist instrument 24.

An outer side of the insertion section 42 is covered with the cylindrical body (sheath) 34 in accordance with an elastic toughness of the flexible portion 52, or details of treatment (air supply, water supply, suction, etc.). The cylindrical body 34 includes the main body (elongated member) 34a and a first engagement portion 74 formed on an outer circumferential surface of the main body 34a along the longitudinal axis L. The main body 34a of the cylindrical body 34 is formed of, for example, a resin material having flexibility. Thus, the elongated member of the insertion instrument 22 includes the cylindrical body 34 having flexibility and provided with the first engagement portion 74. Preferably, a net-like tube (not shown) called a braid is buried in the main body 34a of the cylindrical body 34, so that the main body 34a can be formed to have an appropriate elastic toughness. The main body 34a of the cylindrical body 34 may cover the outer circumferential surface excluding a part that affects a function of the insertion instrument (an observation function in the case of the endoscope 32, or an appropriate treatment function in the case of a treatment instrument) of the distal end 54a of the rigid portion 54.

In a state where no cylindrical body 34 is present, when the distal end of the first engagement portion 74 is supported with the distal end 54a of the rigid portion 54 of the insertion section 42 of the endoscope 32 directed in a vertically upward direction, the direction of the distal end 54a of the rigid portion 54 is widely shifted from the vertically upward direction by the distal section 52a of the flexible portion 52. On the other hand, in a state where the cylindrical body 34 covers an outer periphery of a part including the distal section 52a of the flexible portion 52 of the insertion section 42 of the endoscope 32, the distal end of the first engagement portion 74 is supported with the distal end 54a of the rigid portion 54 of the insertion section 42 of the endoscope 32 directed in the vertically upward direction. At this time, the distal section 52a of the flexible portion 52 causes the distal end 54a of the rigid portion 54 to be shifted from the vertically upward direction because of the presence of the main body 34a of the cylindrical body 34; however, the amount of the shift is largely reduced to several degrees, for example, 10 degrees or less. When an external force other than gravitation is exerted on the rigid portion 54 in a direction shifted from the longitudinal axis L, the distal section 52a of the flexible portion 52 causes the distal end 54a of the rigid portion 54 to be shifted largely away from the vertically upward direction together with the main body 34a of the cylindrical body 34 in accordance with the external force. As the external force other than the gravitation is removed, the distal section 52a of the flexible portion 52, together with the main body 34a of the cylindrical body 34, is returned to a former state in which the external force has not been exerted. Thus, the elongated member has an appropriate elastic toughness by cooperation of the flexible portion 52 and the main body 34a of the cylindrical body 34.

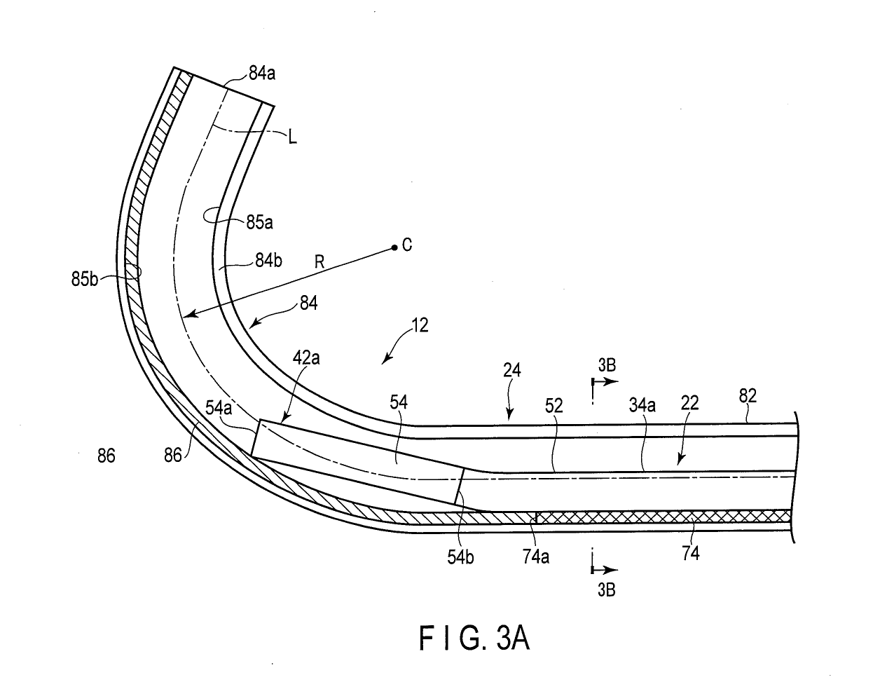

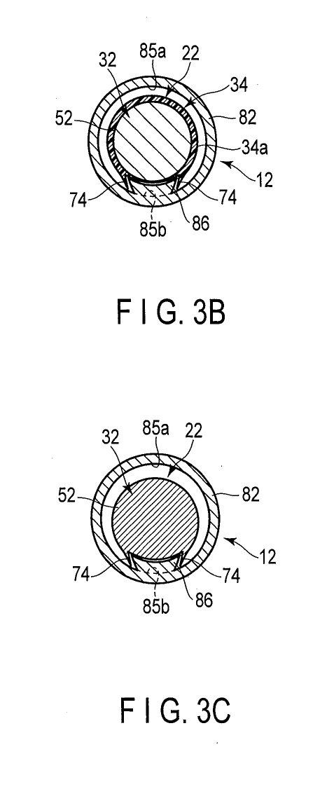

The first engagement portion 74 is preferably a resin material, for example, having flexibility and integrated with the main body 34a of the cylindrical body 34. In this embodiment, the first engagement portion 74 is formed as a concave portion that holds a second engagement portion 86 (to be described later) in a fitted state, as shown in FIG. 3B. The first engagement portion (concave portion) 74 faces a part continuous to a distal part 85b of a bent section 84b to be described later.

The cylindrical body 34 covers the outer circumferential surface of the insertion section 42 of the endoscope 32 over almost the entire length. The cylindrical body 34 covers, in particular, the outer periphery of a boundary between the distal end of the flexible portion 52 and the proximal end of the rigid portion 54 of the insertion section 42 of the endoscope 32. The outer circumferential surface of the distal section 42a of the rigid portion 54 of the insertion section 42 of the endoscope 32 is not necessarily covered.

The first engagement portion 74 is not formed in the distal end of the cylindrical body 34, but is preferably formed on a proximal side from, for example, a position at a distance of several centimeters from the distal end of the cylindrical body 34. Furthermore, the first engagement portion 74 is not necessarily provided to reach the proximal end of the cylindrical body 34, but is preferably formed in only a part of the cylindrical body 34. In any event, when the rigid portion 54 of the endoscope 32 is in the bent section 84b of the guide path 84 of the assist instrument 24 with the endoscope 32 inserted through the cylindrical body 34, a distal end 74a of the first engagement portion 74 is preferably arranged on the proximal side from the proximal end 54b of the rigid portion 54 along the longitudinal axis L. In other words, in a state where the insertion section (insertion main body) 42 of the endoscope 32 is inserted through the cylindrical body 34 and the rigid portion 54 of the distal section 42a of the insertion section 42 is arranged at the distal end of the distal section 34a of the cylindrical body 34, the first engagement portion 74 is provided in a position on the proximal side from the rigid portion 54 of the distal section 42a of the insertion section 42.

The assist instrument (guide member) 24 is continuously formed of a pipe-shaped base portion (guide main body) 82 and the guide path 84. The assist instrument 24 has an inner diameter that allows passage of the insertion section 42 of the endoscope 32 and the main body 34a of the cylindrical body 34. To simplify the description, the base portion 82 is described as a straight pipe. However, the base portion 82 may include an appropriately bent portion. The length of the base portion 82 is determined depending on a target of treatment.

The guide path 84 in this embodiment is pipe-shaped. The guide path 84 includes the distal section 84a and the bent section 84b that is located on a proximal side from the distal section 84a and has a bent shape. The guide path 84 guides the rigid portion 54 of the insertion section 42 of the endoscope 32 to a desired position on a distal side from the distal section 84a (a predetermined position relative to the distal section 84a of the assist instrument 24). The longitudinal axis L is assumed to pass through a central axis of the base portion 82 and to be defined along a bending of the bent section 84b of the guide path 84 and in an extending direction of the distal section 84a.

The distal section 84a of the assist instrument 24 has an inner diameter slightly greater than an outer diameter of the distal section 42a of the insertion section 42 of the endoscope 32, to allow passage of the distal section 42a of the insertion section 42 of the endoscope 32. For example, an inner diameter and a height (a bending radius R to be described later) of the guide path 84 is appropriately set in consideration of operability in handling of the assist instrument 24, in regard to whether or not the assist instrument 24 can be inserted through a target of treatment.

The distal section 84a is preferably formed as an obtuse shape or made of an elastic material, such as rubber. Preferably, the distal section 84a is formed of a material that can easily be deformed upon contact with a living tissue, or capped with a cap-like member. The distal section 84a is also preferably tapered.

Of the inner circumferential surface of the bent section 84b, a side closer to a center C of the bending radius R is referred to as a proximal part 85a and a farther side is referred to as a distal part 85b. In this embodiment, to place the distal section 84a in an appropriate position at an entrance of, for example, a paranasal sinus and to observe and/or treat an inside of the paranasal sinus, the bent section 84b is set at a bending angle of about 110°, which is equal to or greater than 90°, with respect to the base portion 82. The bending angle of the bent section 84b may be appropriately set in accordance with an observation target and/or treatment target, and may be less than 90°.

The guide path 84 is provided with the second engagement portion (engaged portion) 86 engaged with the first engagement portion (engaging portion) 74 of the cylindrical body 34. In this embodiment, the second engagement portion 86 is preferably formed in the distal part 85b, which is opposite to the bent section 84b with respect to the bending direction, of the inner circumferential surface of the bent section 84b of the pipe-shaped guide path 84. Preferably, the second engagement portion 86 is continuously formed in the inner circumferential surface of the distal section 84a, not only the bent section 84b.

Furthermore, each of the first engagement portion 74 and the second engagement portion 86 is described as forming a line here, but of course may preferably forma plurality of parallel lines, for example, two lines.

The second engagement portion 86 is also formed in the base portion 82 beyond the proximal end of the guide path 84. Furthermore, the second engagement portion 86 is preferably formed to be continuous with the guide path 84 and the base portion 82.

In this embodiment, the second engagement portion 86 is formed as a convex portion, as shown in FIG. 3B. The second engagement portion (the convex portion) 86 projects toward a central axis of the pipe-shaped guide path 84. The first engagement portion 74 and the second engagement portion 86 mutually fit each other. Thus, the first engagement portion 74 and the second engagement portion 86 are kept in a state of engagement with each other.

The second engagement portion 86 is relatively slidable relative to the first engagement portion 74 along the longitudinal axis L. Even if the first engagement portion 74 and the second engagement portion 86 are moved in a direction crossing the axial direction, the movement is restricted. Therefore, the state of engaging the first engagement portion 74 with the second engagement portion 86 is maintained.

Here, the distal end 74a of the first engagement portion 74 is located on a proximal side with an appropriate distance from the proximal end 54b of the rigid portion 54 along the longitudinal axis L in the cylindrical body 34. Therefore, in a state where the distal end 74a of the first engagement portion 74 is supported or bound by the second engagement portion 86, the distal section 52a (a part between the distal end 74a of the first engagement portion 74 and the proximal end 54b of the rigid portion 54) of the flexible portion 52 can be bent in an appropriate state.

As described above, the main body 34a of the cylindrical body 34 covering the outer periphery of the insertion section 42 of the endoscope 32 has an appropriate elastic toughness, and covers the flexible portion 52 of the insertion section 42 of the endoscope 32 in a part on a distal side from the distal end 74a of the first engagement portion 74. Of the distal section 52a of the flexible portion 52 and the main body 34a of the cylindrical body 34, the part covering the distal section 52a of the flexible portion 52 is arranged along the shape of the distal part 85b of the guide path 84 or substantially along the shape of the distal part 85b in a state where an external force other than gravitation is not exerted on the rigid portion 54. Therefore, the distal section 52a of the flexible portion 52 of the insertion section 42 of the endoscope 32 according to the embodiment is supported by the main body 34a of the cylindrical body 34, and prevented from buckling by the gravitation. On the other hand, when an external force is exerted on the rigid portion 54 from a direction shifted from the longitudinal axis L, the part covering the distal section 52a of the flexible portion 52, of the distal section 52a of the flexible portion 52 on the distal side from the distal end 74a of the first engagement portion 74 and the main body 34a of the cylindrical body 34, is appropriately bent in accordance with the external force.

Next, an operation of the treatment system 10 according to the embodiment will be described. Here, an example of arranging the distal end 54a of the insertion instrument 22 at a predetermined location relative to the distal section 84a of the assist instrument 24 using the insertion assist system 12 will be described.

The first engagement portion 74, provided in the main body 34a of the cylindrical body 34, is engaged with the second engagement portion 86 extending along the longitudinal axis L of the base portion 82 and the guide path 84 of the assist instrument 24. Thus, the distal section 84a of the guide path 84 of the assist instrument 24 is directed upward as indicated by a solid line shown in FIG. 1, and the rigid portion 54 of the insertion section 42 of the endoscope 32 is projected as shown in FIG. 1.

As an example, the rigid portion 54 of the insertion section 42 of the endoscope 32 is guided from the proximal end of the assist instrument 24 toward the distal end. At this time, as shown in FIG. 3A and FIG. 3B, the first engagement portion 74 and the second engagement portion 86 are engaged (fit). Therefore, when the first engagement portion 74 and the second engagement portion 86 are slid relative to each other along the longitudinal axis L, the outer circumferential surface of the main body 34a of the cylindrical body 34 is kept in contact with or in proximity to the base portion 82. Then, as shown in FIG. 3A, the rigid portion 54 of the insertion section 42 of the endoscope 32 is guided through the base portion 82 of the assist instrument 24 to the bent section 84b of the guide path 84.

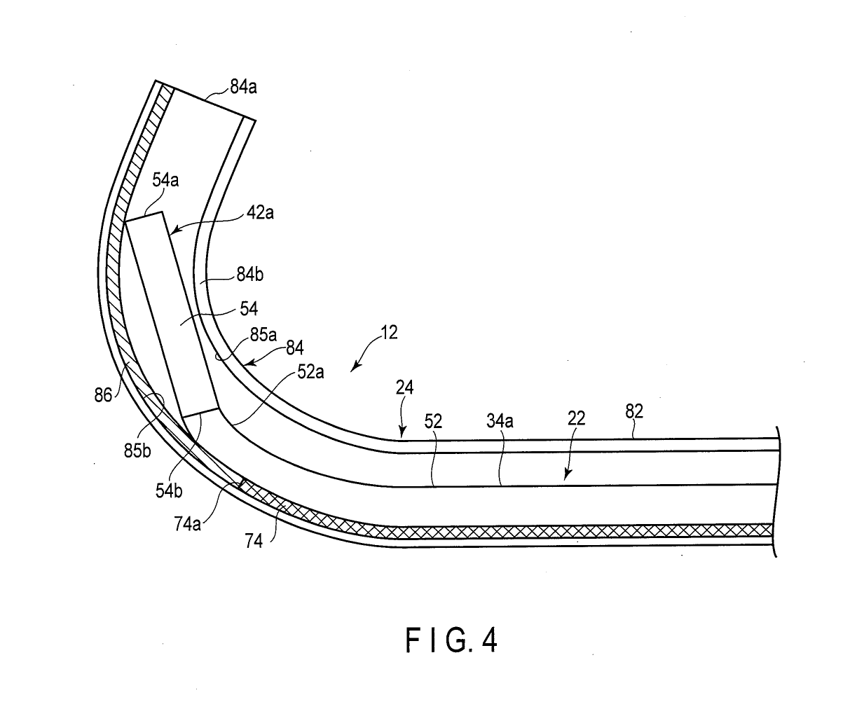

The distal end 54a of the rigid portion 54 of the insertion section 42 of the endoscope 32 is moved from a position in proximity to the base portion 82 (see FIG. 3A) of the bent section 84b of the guide path 84 of the assist instrument 24 to a position in proximity to the distal section 84a of the guide path 84 (see FIG. 4).

At this time, the insertion section 42 of the endoscope 32 is engaged with the first engagement portion 74 of the assist instrument 24 by the second engagement portion 86 at a position on a proximal side from a boundary between the proximal end 54b of the rigid portion 54 and the distal end of the flexible portion 52. Therefore, the direction of the distal end 54a of the rigid portion 54 changes in accordance with a shape of the distal part 85b of the bent section 84b, while the distal end 54a is being in contact with the second engagement portion 86 (the distal part 85b) of the bent section 84b.

At this time, only the distal end 54a of the rigid portion 54 is in contact with the second engagement portion 86 (the distal part 85b) of the bent section 84b. In other words, the distal end 54a of the rigid portion 54 receives a reaction force due to contact from the second engagement portion 86 (the distal part 85b) of the bent section 84b. Furthermore, it is assumed that an appropriate load may be applied to the proximal end 54b of the rigid portion 54 by the distal section 52a of the flexible portion 52, as the direction of the distal end 54a of the rigid portion 54 changes. However, as shown in FIG. 3A and FIG. 4, the rigid portion 54 is prevented from being brought into contact with the proximal part 85a of the bent section 84b. Therefore, application of a load to a part between the distal end 54a and the proximal end 54b of the rigid portion 54 is suppressed. Thus, only the reaction force from the distal part 85b of the bent section 84b is applied as a load to the rigid portion 54, while application of a reaction force from the proximal part 85a is prevented. Accordingly, only a force that supports the distal end 54a and the proximal end 54b of the rigid portion 54 is applied to the rigid portion 54. A force that presses an appropriate part between the distal end 54a and the proximal end 54b of the rigid portion 54 from the proximal part 85a of the bent section 84b toward the distal part 85b of the bent section 84b is prevented from being applied as a load. Thus, a load that may bend the straight rigid portion 54 is prevented from being applied to the rigid portion 54. In other words, in this embodiment, a load that may be subjected to a so-called three-point bending test for material, such as metallic material, is prevented from being applied to the rigid portion 54.

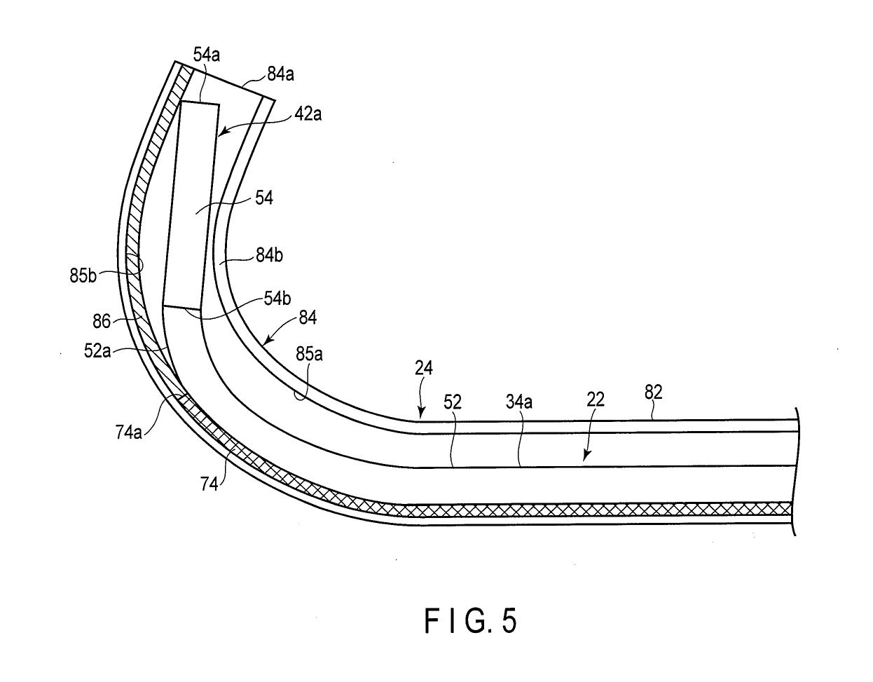

Then, for example, while the distal end 54a of the insertion section 42 of the endoscope 32 is located at a position relative to the assist instrument 24 as shown in FIG. 5, the insertion section 42 of the endoscope 32 and the main body 34a of the cylindrical body 34 are supported by the movable portion 26b of the handle 26. At this time, the movable portion 26b is located at the most proximal end along the longitudinal axis L of the handle main body 26a shown in FIG. 1.

When the movable portion 26b is moved forward along the longitudinal axis L of the handle main body 26a, the cylindrical body 34, through which the insertion section 42 of the endoscope 32 passes, moves toward a distal side along the longitudinal axis L relative to the base portion 82 and the guide path 84 of the assist instrument 24 together with the insertion section 42 of the endoscope 32.

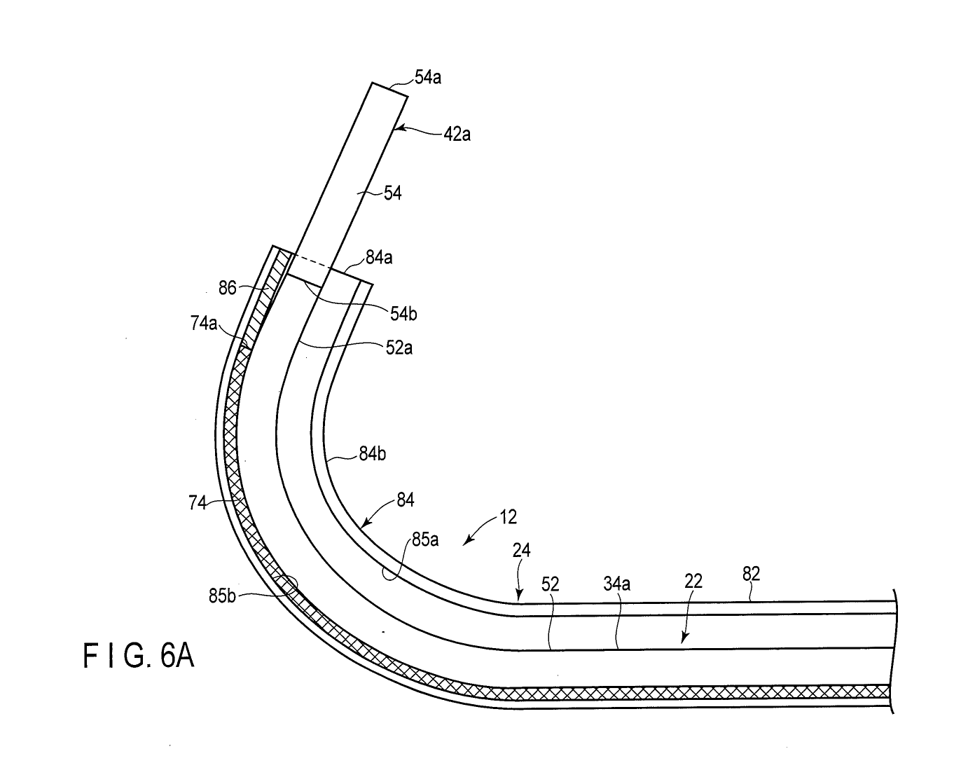

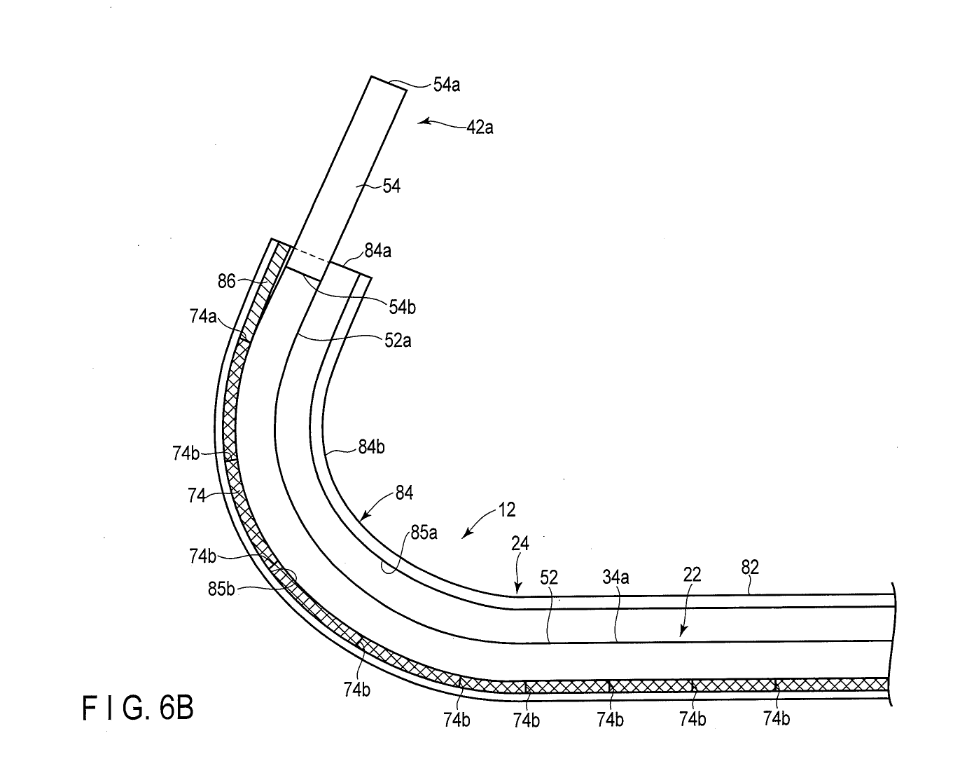

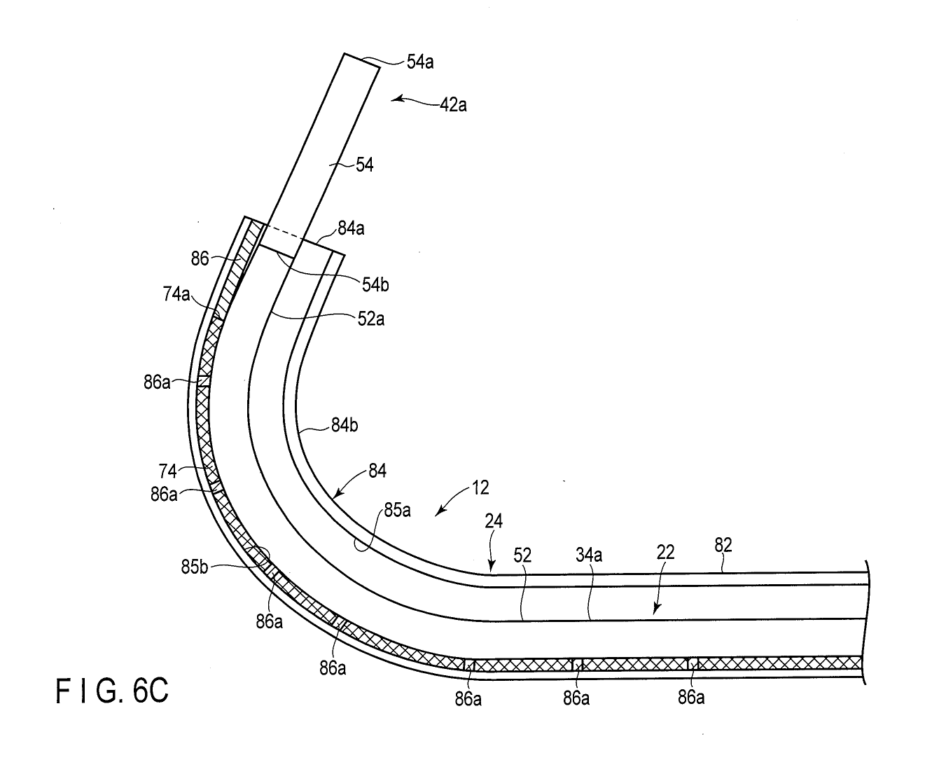

Then, as shown in FIG. 6A, the distal end 54a of the rigid portion 54 of the insertion section 42 of the endoscope 32 is projected from the distal section 84a of the guide path 84 of the assist instrument 24.

The distal section 84a of the assist instrument 24 is directed upward. Therefore, gravitation is applied to the rigid portion 54 of the insertion section 42 of the endoscope 32 as an external force. In the insertion section 42 of the endoscope 32, the distal section 52a of the flexible portion 52 is covered with the main body 34a of the cylindrical body 34. At this time, the distal end 54a of the rigid portion 54 counteracts gravitation due to the elastic toughness of the distal section 52a of the flexible portion 52 of the insertion section 42 and the main body 34a of the cylindrical body 34 of the endoscope 32 described above. Therefore, the distal end 54a of the rigid portion 54 is prevented from wobbling relative to the distal section 84a of the guide path 84 of the assist instrument 24. Accordingly, the rigid portion 54 of the insertion section 42 or the distal section 52a of the flexible portion 52 of the endoscope 32 are in contact with or in proximity to the second engagement portion 86 (the distal part 85b) of the distal section 84a of the assist instrument 24. Consequently, even if the bent section 84b of the guide path 84 is set at a bending angle equal to or greater than 90° with respect to the base portion 82, the rigid portion 54 of the insertion section 42 of the endoscope 32 is maintained in proximity to the second engagement portion 86 (the distal part 85b) of the distal section 84a of the guide path 84 of the assist instrument 24.

Thus, when the distal end 54a of the rigid portion 54 is projected from the distal section 84a of the assist instrument 24, the insertion section 42 of the endoscope 32 of this embodiment is maintained in line with the second engagement portion 86 in the distal section 84a of the assist instrument 24. The distal end 54a of the rigid portion 54 of the insertion section 42 of the endoscope 32 is arranged at a predetermined location, in a state of projecting from the distal section 84a of the assist instrument 24. Therefore, when the distal end 54a of the rigid portion 54 is projected from the distal section 84a of the guide path 84, the distal end 54a of the rigid portion 54 is guided to a predetermined position. Specifically, the first engagement portion 74 and the second engagement portion 86, in association with each other, maintain the flexible portion (elongated member) 52 of the insertion section 42 of the endoscope 32 and the guide path 84 in contact with, or in proximity to the guide path 84, and guide the rigid portion 54 of the insertion section 42 of the endoscope 32 to a predetermined position in a distal side from the distal section 84a of the guide path 84 of the assist instrument 24.

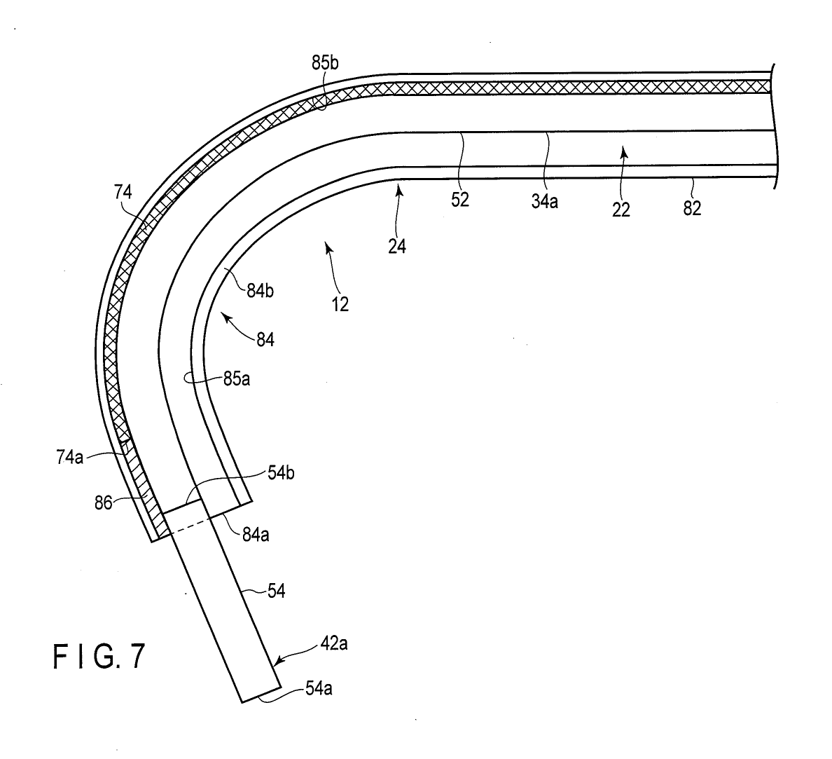

As shown in FIG. 7, the distal section 84a of the assist instrument 24 may be directed downward (a state indicated by broken lines in FIG. 1). At this time, a direction of gravitation relative to the insertion instrument 22 and the assist instrument 24 is changed. To maintain the state in which the first engagement portion 74 and the second engagement portion 86 are engaged, the insertion section 42 of the endoscope 32 is arranged in the base portion 82, the bent section 84b of the guide path 84, and the distal part 85b of the distal section 84a. Therefore, even if gravitation is applied to the insertion section 42 of the endoscope 32 as a load, the distal end 54a of the rigid portion 54 of the insertion section 42 of the endoscope 32 is arranged at a predetermined location relative to the distal section 84a, in a state of projecting from the distal section 84a of the assist instrument 24.

A part of the distal section 52a of the flexible portion 52 of the insertion section 42 of the endoscope 32 in combination with a part covering the distal section 52a of the flexible portion 52 of the main body 34a of the cylindrical body 34 (a distal section of the elongated member) has an appropriate elastic toughness (flexural rigidity against an external force and restorability from a bent state). Although not shown in the drawings, when the base portion 82 of the assist instrument 24 is rotated around the longitudinal axis L, the distal section 84a of the assist instrument 24 is directed to a point between the positions of the examples shown in FIG. 6A and FIG. 7A. In this state, even when gravitation is applied as a load to the rigid portion 54 of the insertion section 42 of the endoscope 32, as shown in FIG. 6A and FIG. 7, the distal end 54a of the rigid portion 54 of the insertion section 42 of the endoscope 32 is arranged at a predetermined location relative to the distal section 84a in the state of projecting from the distal section 84a of the assist instrument 24, due to an appropriate elastic toughness (flexural rigidity) in the part of the distal section 52a of the flexible portion 52 in combination with the part of the main body 34a of the cylindrical body 34 (the distal section of the elongated member).

When an external force other than gravitation is applied to the rigid portion 54 of the insertion section 42 of the endoscope 32 shown in FIG. 6A and FIG. 7, a part covering the distal section 52a of the flexible portion 52, of the distal section 52a of the flexible portion 52 and the main body 34a of the cylindrical body 34, is bent in accordance with the external force. When external force other than gravitation is removed, the distal end 54a of the rigid portion 54 of the insertion section 42 of the endoscope 32 is returned to a predetermined position relative to the distal section 84a in the state of projecting from the distal section 84a of the assist instrument 24, due to an appropriate elastic toughness (restorability from a bent state) in the part of the distal section 52a of the flexible portion 52 in combination with the part of the main body 34a of the cylindrical body 34 (a distal section of the elongated member).

An elastic toughness in the distal section 52a of the flexible portion 52 in combination with the main body 34a of the cylindrical body 34 (a distal section of the elongated member) can be appropriately set. Therefore, when external force other than gravitation is applied as a load, the distal end 54a of the rigid portion 54 of the insertion section 42 of the endoscope 32 need not be returned to a state at a position relative to the distal section 84a while projecting from the distal section 84a of the assist instrument 24, depending on a state of observation or treatment.

The distal section of the elongated member in the description may include or may not include the distal end 74a of the first engagement portion 74 by adjusting elastic deformability of the first engagement portion 74.

As described above, the embodiment acts as follows:

In a case of guiding the distal end 54a of the insertion instrument 22 including the rigid portion 54 to a desired position through the guide path 84 that includes the bent part (the bent section 84b) of the assist instrument 24, the first engagement portion 74 of the insertion instrument 22 is engaged with the second engagement portion 86 of the assist instrument 24. Then, when the rigid portion 54 of the insertion instrument 22 is passed through the bent section 84b of the guide path 84 of the assist instrument 24, the rigid portion 54 is prevented from being brought into contact with the proximal part 85a that faces a surface on which the second engagement portion 86 of the assist instrument 24 is formed. Therefore, when the elongated insertion instrument 22 including the rigid portion 54 is inserted through the assist instrument 24 from the proximal end toward the distal end, a load can be prevented from being applied to the rigid portion 54. If the insertion instrument 22 is the endoscope 32, a load is prevented from being applied in a direction in which the rigid portion 54 is bent from the axial direction. Since a load is prevented from being applied to the rigid portion 54, axis deviation of an optical axis of the observation optical system 56 of the endoscope 32 is also prevented.

Furthermore, as described above, when the distal end 54a of the rigid portion 54 is projected from the distal section 84a of the assist instrument 24, the state of engagement of the first engagement portion 74 and the second engagement portion 86 is maintained. Therefore, even if gravitation is appropriately applied to the rigid portion 54 of the insertion section 42 of the endoscope 32 as a load, the distal end 54a of the rigid portion 54 of the insertion section 42 of the endoscope 32 can be arranged at a predetermined location relative to the distal section 84a of the assist instrument 24, in a state of projecting from the distal section 84a of the assist instrument 24.

In a case of inserting the insertion section 42 of the endoscope 32 that has a small outer diameter into a desired part, the distal end 54a of the insertion section 42 of the endoscope 32 is required to move forward and backward at a desired angle relative to the distal section 84a of the assist instrument 24. In a case of using the insertion assist system 12 of this embodiment, the distal end 54a of the insertion section 42 of the endoscope 32 can be directed to a desired direction.

This embodiment is effective in a case where an inner diameter of the pipe-shaped guide path 84 (greater than the outer diameter of the rigid portion 54) is as small as possible, relative to the rigid portion 54 having an appropriate length on the distal side of the flexible portion 52, while the guide path 84 allows passage of the rigid portion 54. Thus, the inner diameter of the guide path 84 can be as small as possible by using the first engagement portion 74 and the second engagement portion 86 of this embodiment described above.

In the case of a treatment instrument that has an elongated member having flexibility and extending along the longitudinal axis and the rigid portion 54 provided on a distal side of the elongated member, the first engagement portion 74 of the elongated member and the second engagement portion 86 of the assist instrument 24 are appropriately engaged. Therefore, the distal end 54a of the rigid portion 54 of the insertion section 42 of the treatment instrument can be arranged at a predetermined location relative to the distal section 84a of the assist instrument 24 in a state of projecting from the distal section 84a of the assist instrument 24.

As shown in FIG. 2 and FIG. 3B, this embodiment is described as an example in which the cylindrical body (sheath) 34 is arranged at an outer periphery of the insertion section 42 of the endoscope 32. However, as shown in FIG. 3C, the cylindrical body 34 may not be present. At this time, the elongated member of the insertion instrument 22 is the flexible portion 52 having flexibility, extending along the longitudinal axis L, and provided with the first engagement portion 74.

In this case, it is preferable that the rigidity of the flexible portion 52, in particular an outer surface of the distal section 52a, is adjusted to appropriately adjust the flexibility and the elastic toughness of the flexible portion 52. For example, the distal end 74a of the first engagement portion 74 is supported with the distal end 54a of the rigid portion 54 of the insertion section 42 of the endoscope 32 directed in the vertically upward direction. At this time, the distal section 52a of the flexible portion 52 has an elastic toughness in its peripheral portion in the same manner as in the case where the main body 34a of the cylindrical body 34 is present. Therefore, the distal end 54a of the rigid portion 54 is prevented from being deviated from the vertically upward direction. When an external force other than gravitation is exerted on the rigid portion 54 in a direction shifted from the longitudinal axis L, the distal section 52a of the flexible portion 52 causes the distal end 54a of the rigid portion 54 to be shifted largely away from the vertically upward direction in accordance with the external force. Furthermore, when the external force other than the gravitation is removed, the distal section 52a of the flexible portion 52 is preferably returned to a former state in which the external force has not been exerted.

Thus, when the distal end 54a of the rigid portion 54 is projected from the distal section 84a of the assist instrument 24 as shown in FIG. 6A, the insertion section 42 of the endoscope 32 is maintained in line with the second engagement portion 86 in the distal section 84a of the assist instrument 24. The distal end 54a of the rigid portion 54 of the insertion section 42 of the endoscope 32 is arranged at a predetermined location, in a state of projecting from the distal section 84a of the assist instrument 24. Therefore, when the distal end 54a of the rigid portion 54 is projected from the distal section 84a of the guide path 84, the distal end 54a of the rigid portion 54 of the insertion instrument 22 is guided to a predetermined position.

Moreover, regardless of whether the cylindrical body 34 is present or not, a load is prevented from being applied to the rigid portion 54 of the endoscope 32 in a direction in which the rigid portion 54 is bent from the axial direction. Since a load is prevented from being applied to the rigid portion 54, axis deviation of an optical axis of the observation optical system 56 of the endoscope 32 is also prevented.

The embodiment has been described in connection with a case in which the first engagement portion 74 is continuously formed, as shown in FIG. 6A and FIG. 7. In this case, since the first engagement portion 74 is formed as a continuous ridge, the flexibility of the main body 34a of the cylindrical body 34 (the flexible portion 52 in the case shown in FIG. 3C) in a part on a proximal side from the distal end of the first engagement portion 74 may be lowered depending on a selected material, size, etc. It is also preferable that the first engagement portion 74 is discretely formed along the longitudinal axis L, as shown in FIG. 6B. Here, slits 74b are formed in the first engagement portion 74. Therefore, the first engagement portion 74 is more bendable along the shape of the distal part 85b of the guide path 84 as compared to the state shown in FIG. 6A. Since the distal end of the main body 34a of the cylindrical body 34 (the flexible portion 52 in the example shown in FIG. 3C) is distant from the distal end 74a of the first engagement portion 74, there is little or no influence upon the elastic toughness of the distal section 52a of the flexible portion 52.

Furthermore, the embodiment has been described in connection with a case in which the second engagement portion 86 is continuously formed, as shown in FIG. 6A and FIG. 7. It is also preferable that the second engagement portion 86 is discretely formed along the longitudinal axis L, as shown in FIG. 6C. Here, slits 86a are formed in the second engagement portion 86. The second engagement portion 86 is not necessarily continuous, as long as the state of engagement with the first engagement portion 74 is maintained.

The first engagement portion 74 including the slits 74b as shown in FIG. 6B can of course be engaged with the second engagement portion 86 including the slits 86a as shown in FIG. 6C.

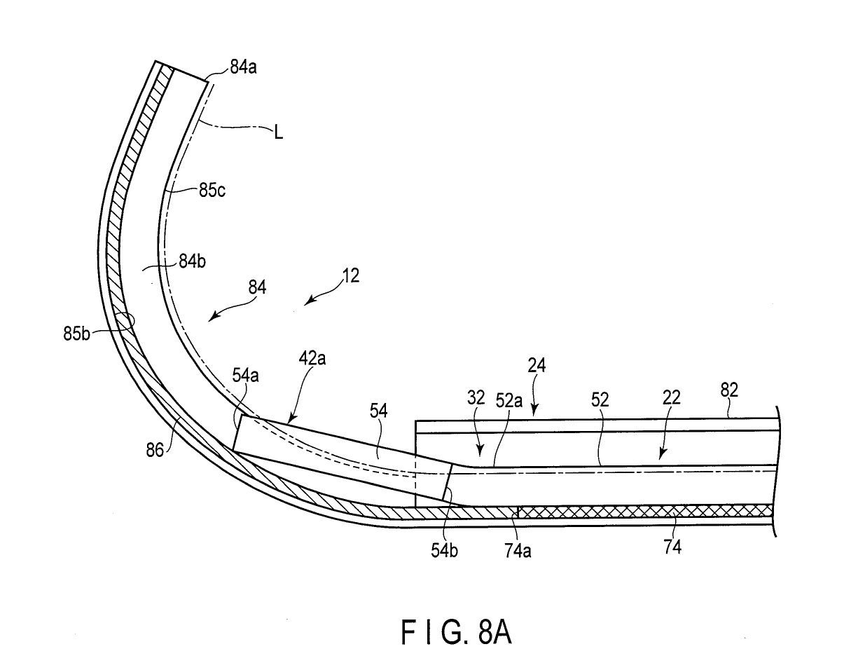

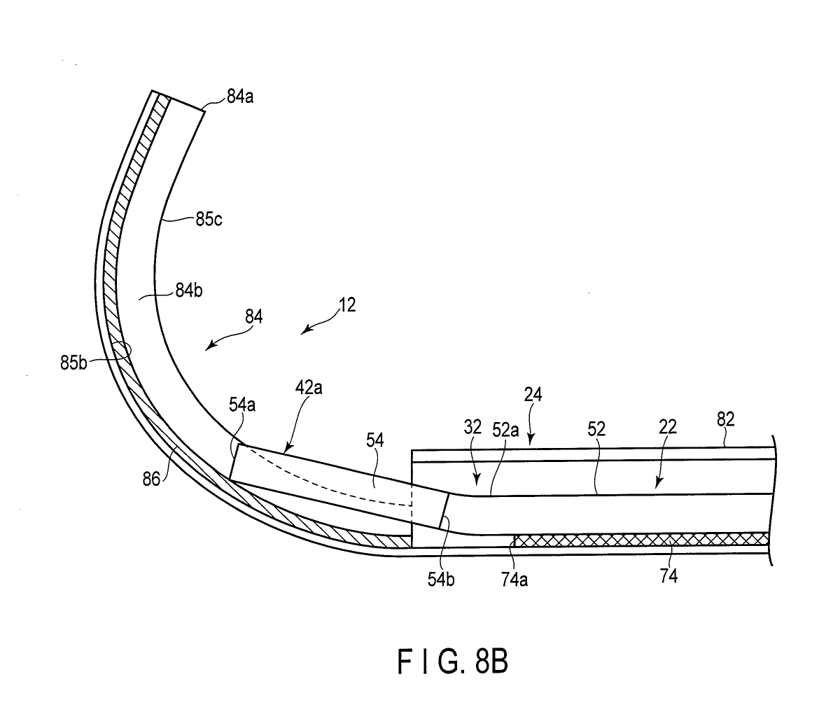

A second embodiment will be described with reference to FIG. 8A to FIG. 10. This embodiment is a modification of the first embodiment. The same members or the members having the same functions as those of the members of the first embodiment are identified by the same reference symbols as those used for the first embodiment as much as possible, and detailed explanations thereof are omitted.

An explanation will be given for a case as shown in FIG. 8A, in which a guide path 84 has a half pipe-like shape or an almost-flat spatula shape, not a pipe-like shape. For example, a pair of edge portions 85c that are preferably parallel to the longitudinal axis L are formed in a bent section 84b of the guide path 84. The pair of edge portions 85c are preferably formed as obtuse shapes.

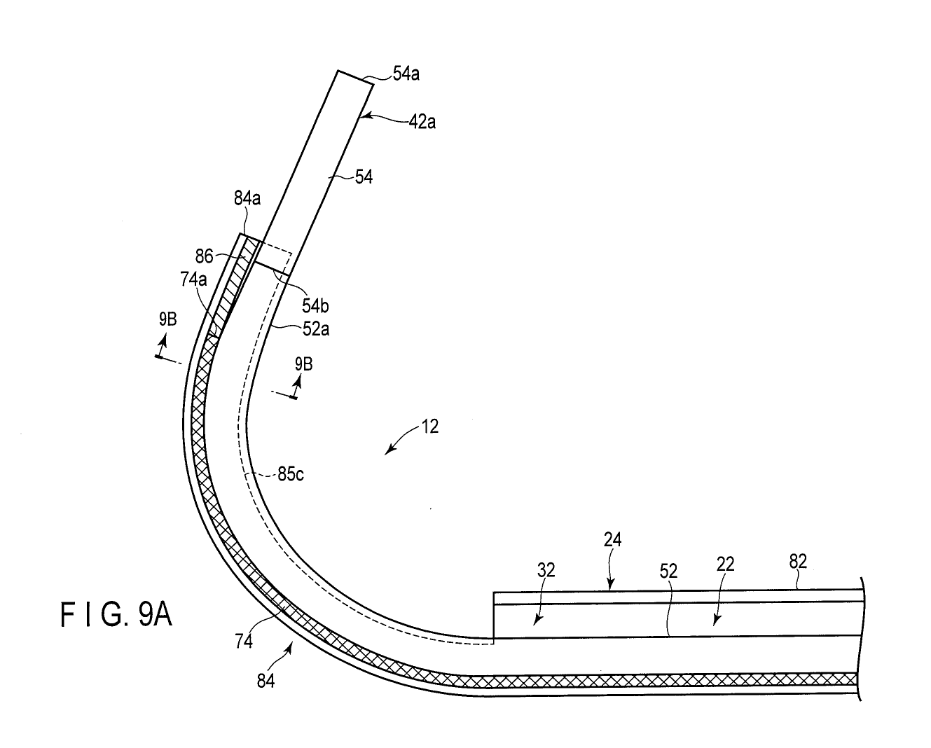

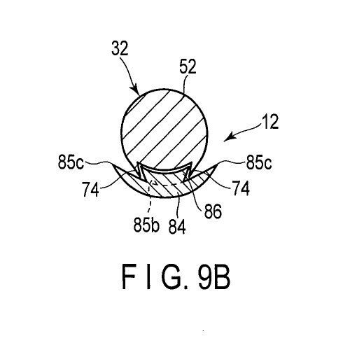

As shown in FIG. 9B, even if the guide path 84 has a half pipe-like shape, the state of engagement between a first engagement portion 74 and a second engagement portion 86 can be maintained in the same manner as that described in connection with the first embodiment. Thus, as shown in FIG. 9A, when a distal end 54a of a rigid portion 54 is projected from a distal section 84a of an assist instrument 24, an insertion section 42 of an endoscope 32 of this embodiment is maintained in line with the second engagement portion 86 in the distal section 84a of the assist instrument 24. Therefore, the distal end 54a of the rigid portion 54 of the insertion section 42 of the endoscope 32 is arranged at a predetermined location, in a state of projecting from the distal section 84a of the assist instrument 24. Consequently, when the distal end 54a of the rigid portion 54 is projected from the distal section 84a of the guide path 84, the distal end 54a of the rigid portion 54 is guided to a predetermined location.

This embodiment is suitable for arranging the distal end 54a of the rigid portion 54 at a predetermined location projecting from the distal section 84a of the guide path 84, while the rigid portion 54 having an appropriate length on a distal side of the flexible portion 52 is not separated from the spatula guide path 84 to the greatest extent. In particular, since a proximal part 85a on an inner circumferential surface of the bent section 84b is not present, the rigid portion 54 cannot be brought into contact with a proximal part 85a on the inner circumferential surface of the bent section 84b. Therefore, in this embodiment, a bending radius R of a distal part 85b of the bent section 84b of the guide path 84 can be not only enlarged, but also reduced as compared to the case of the first embodiment described above.

Furthermore, the second engagement portion 86 need not be formed in a base portion 82, while the second engagement portion 86 is formed in the guide path 84 of the assist instrument 24, as shown in FIG. 8B. Even in this case, as shown in FIG. 9A, it is only necessary that the first engagement portion 74 and the second engagement portion 86 be engaged, when the distal end 54a of the insertion section 42 of the endoscope 32 is projected from the distal section 84a of the guide path 84.

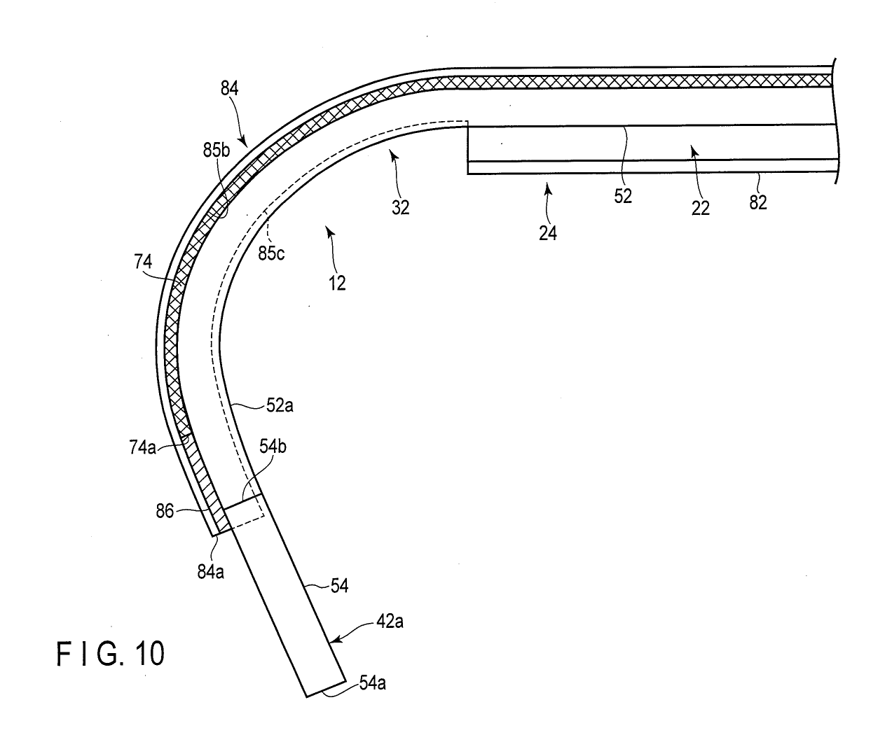

As shown in FIG. 10, the distal section 84a of the assist instrument 24 may be directed downward (a state indicated by broken lines in FIG. 1). To maintain the state in which the first engagement portion 74 and the second engagement section 86 are engaged, the insertion section 42 of the endoscope 32 is arranged in the base portion 82, the bent section 84b of the guide path 84, and the distal part 85b of the distal section 84a. Therefore, even if gravitation is applied to the insertion section 42 of the endoscope 32 as a load, the distal end 54a of the rigid portion 54 of the insertion section 42 of the endoscope 32 is arranged at a predetermined location, in a state of projecting from the distal section 84a of the assist instrument 24.

The relationship between the first engagement portion 74 and the second engagement portion 86 is not limited to the examples in the first embodiment and the second embodiment described above.

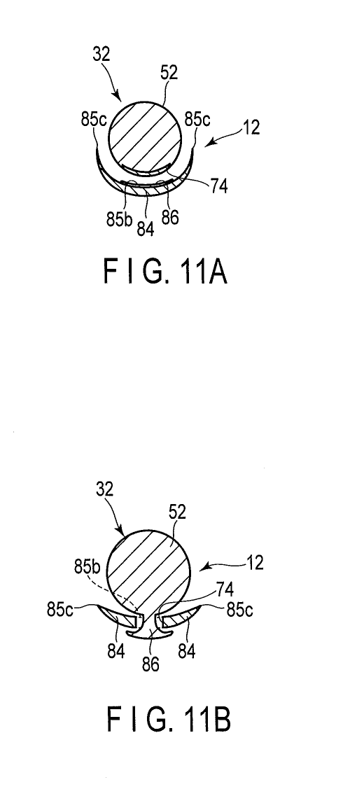

For example, as shown in FIG. 11A, a permanent magnet or a ferromagnetic body is used as the first engagement portion 74. The first engagement portion 74 may be formed continuously or discretely at appropriate intervals on a proximal side in a part not including the distal section 52a of the flexible portion 52 of the insertion instrument 22.

For example, a permanent magnet or a ferromagnetic body is used as the second engagement portion 86. If a ferromagnetic body is used as the first engagement portion 74, a permanent magnet is used as the second engagement portion 86. Thus, it is only necessary that the first engagement portion 74 and the second engagement portion 86 are in a condition that appropriate attractive forces act on each other. Specifically, one of the first engagement portion 74 and the second engagement portion 86 includes a magnet, while the other of the first engagement portion 74 and the second engagement portion 86 includes another magnet that exerts an attractive force on the aforementioned magnet or includes a ferromagnetic body that is attracted to the aforementioned magnet with an attraction force.

Even in the case where the first engagement portion 74 and the second engagement portion 86 are thus formed, when the distal end 54a of the rigid portion 54 is projected from the distal section 84a of the assist instrument 24, the insertion section 42 of the endoscope 32 is maintained in line with the second engagement portion 86 in the distal section 84a of the assist instrument 24. Thus, the distal end 54a of the rigid portion 54 of the insertion section 42 of the endoscope 32 is arranged at a predetermined location, in a state of projecting from the distal section 84a of the assist instrument 24. Consequently, when the distal end 54a of the rigid portion 54 is projected from the distal section 84a of the guide path 84, the distal end 54a of the rigid portion 54 is guided to a predetermined location. At this time, the flexible portion (elongated member) 52 of the insertion section 42 of the endoscope 32 and the guide path 84 of the assist instrument 24 are maintained in a contact state.

For example, as shown in FIG. 11B, a projecting portion having a nearly T-shaped cross section is used as the first engagement portion 74. If the first engagement portion 74 is continuously formed from the distal end of the first engagement portion 74 to a proximal side of the insertion instrument 22, the first engagement portion 74 has flexibility for appropriately deforming in accordance with a bending state of the bent section 84b. Even if a short first engagement portion 74 is formed along the longitudinal axis L, it is preferable that flexibility is provided.

Even in the case where the first engagement portion 74 and the second engagement portion 86 are thus formed, when the distal end 54a of the rigid portion 54 is projected from the distal section 84a of the assist instrument 24, the insertion section 42 of the endoscope 32 is maintained in line with the second engagement portion 86 in the distal section 84a of the assist instrument 24. Thus, the distal end 54a of the rigid portion 54 of the insertion section 42 of the endoscope 32 is arranged at a predetermined location, in a state of projecting from the distal section 84a of the assist instrument 24. Consequently, when the distal end 54a of the rigid portion 54 is projected from the distal section 84a of the guide path 84, the distal end 54a of the rigid portion 54 is guided to a predetermined location. At this time, the flexible portion (elongated member) 52 of the insertion section 42 of the endoscope 32 and the guide path 84 of the assist instrument 24 are maintained in a contact state.

The examples of the guide path 84 shown in FIG. 11A and FIG. 11B having a half pipe-like shape and a spatula shaped are described. However, the guide path 84 may of course have a pipe-like shape. Therefore, the first engagement portion 74 and the second engagement portion 86 shown in FIG. 11A are applicable to a guide path 84 that has a pipe-like shape. Similarly, the first engagement portion 74 and the second engagement portion 86 shown in FIG. 11B are applicable to a guide path 84 that has a pipe-like shape.

In the above descriptions of the first embodiment and the second embodiment, to simplify the explanation, the bent section 84b of the guide path 84 is described as being bent in only one direction with respect to the base portion 82. The bent section 84b of the guide path 84 may be appropriately bent in anyway with respect to the base portion 82. Specifically, the guide path 84 may be winding. Thus, not all of the bent section 84b need be bent. In other words, at least a part of the bent section 84b may be bent.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An insertion assist system comprising:
an insertion instrument including an elongated member having flexibility and extending along a longitudinal axis, a first engagement portion provided in the elongated member, and a rigid portion provided on a distal side of the elongated member and having an appropriate length; and
an assist instrument including a guide path that includes a distal section, and a bent section, at least a part of the bent section being bent at a position on a proximal side from the distal section, and that is configured to guide the rigid portion of the insertion instrument to a desired position on a distal side from the distal section, and a second engagement portion that is provided in the guide path and engaged with the first engagement portion of the insertion instrument, and that maintains the elongated member and the guide path in a contact or close state.

2. The insertion assist system according to claim 1, wherein the first engagement portion is slidable relative to the second engagement portion along the longitudinal axis.

3. The insertion assist system according to claim 2, wherein:
one of the first engagement portion and the second engagement portion includes a convex portion continuously or discretely formed along the longitudinal axis; and
another of the first engagement portion and the second engagement portion includes a concave portion continuously or discretely formed along the longitudinal axis and engaged with the convex portion.

4. The insertion assist system according to claim 1, wherein a distal end of the first engagement portion is located on a proximal side from a proximal end of the rigid portion in the insertion instrument.

5. The insertion assist system according to claim 1, wherein the second engagement portion is provided in the bent section.

6. The insertion assist system according to claim 1, wherein:
one of the first engagement portion and the second engagement portion includes a magnet; and
another of the first engagement portion and the second engagement portion includes another magnet that exerts an attractive force on the magnet or a ferromagnetic body that is attracted to the magnet by an attractive force.

7. The insertion assist system according to claim 1, wherein the elongated member of the insertion instrument includes a cylindrical body having flexibility and provided with the first engagement portion.

8. The insertion assist system according to claim 7, wherein:
the elongated member of the insertion instrument includes a flexible portion extending along the longitudinal axis within the cylindrical body; and
the insertion instrument includes an endoscope including the cylindrical body, the flexible portion, and the rigid portion.

9. The insertion assist system according to claim 1, wherein:
the elongated member of the insertion instrument includes a flexible portion having flexibility, extending along the longitudinal axis, and provided with the first engagement portion; and
the insertion instrument includes an endoscope including the flexible portion and the rigid portion.

10. The insertion assist system according to claim 1, wherein the guide path of the assist instrument has a pipe-like shape.

11. The insertion assist system according to claim 1, wherein the guide path of the assist instrument has a spatula shape.

12. The insertion assist system according to claim 1, the distal section of the assist instrument is formed as an obtuse shape or made of an elastic material.

13. An insertion assist system comprising:
an insertion instrument including:
an insertion main body including a flexible tube having flexibility and extending along a longitudinal axis, and a rigid portion provided on a distal side of the flexible tube and having an appropriate length,
a cylindrical body through which the insertion main body is inserted, and which is provided on an outer circumferential surface of the insertion main body and forms an elongated member having an appropriate elastic toughness in association with the flexible tube, and
a first engagement portion provided at a position on a proximal side from the rigid portion in the cylindrical body in a state in which a distal section of the insertion main body is arranged at a distal end of the cylindrical body; and
an assist instrument including a guide path that includes a distal section and a bent section, at least apart of the bent section being bent at a position on a proximal side from the distal section, and that is configured to guide the rigid portion to a desired position on a distal side from the distal section, the assist instrument including a second engagement portion that is provided in the guide path and engaged with the first engagement portion, and maintains the cylindrical body and the guide path in a contact or close state.

14. The insertion assist system according to claim 13, wherein the cylindrical body continuously covers at least a part of an outer circumferential surface of the rigid portion and a distal section of the flexible tube.

15. An insertion instrument comprising:
an elongated member having flexibility and guided along an assist instrument provided with an engaged portion to a guide path extending along a longitudinal axis;
an engagement portion provided in the elongated member and configured to move the elongated member relative to the assist instrument along the longitudinal axis in a state of being engaged with the engaged portion; and
a rigid portion provided on a distal side of the elongated member, having an appropriate length, and guided to a predetermined position relative to a distal section of the guide path on a distal side from the distal section of the guide path in a state where the engagement portion is engaged with the engaged portion to bring the elongated member and the guide path in a contact or close state.

16. The insertion instrument according to claim 15, wherein the engagement portion is slidable along the longitudinal axis in a state of being engaged with the engaged portion.

17. The insertion instrument according to claim 15, wherein the elongated member includes a cylindrical body having flexibility and provided with the engagement portion.

18. The insertion instrument according to claim 17, wherein:
the elongated member includes a flexible portion extending along the longitudinal axis within the cylindrical body; and
the insertion instrument includes the cylindrical body, and an endoscope including the flexible portion and the rigid portion.

19. The insertion instrument according to claim 15, wherein:
the elongated member includes a flexible portion having flexibility, extending along the longitudinal axis, and provided with the engagement portion; and
the insertion instrument includes an endoscope including the flexible portion and the rigid portion.
